(19) United States
(12) Patent Application Publication
(10) Pub. No.: US 2007/0004774 A1
(43) Pub. Date: Jan. 4, 2007

(54) Cept inhibitors and metabolites thereof

(75) Inventors: Deepak Kamalnath Dalvie, San Diego, CA (US); Roger Benjamin Ruggeri, San Diego, CA (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/579,898

(22) PCT Filed: Sep. 30, 2004
(86) PCT No.: PCT/IB04/03054

§ 371 Date: May 19, 2006

Related U.S. Application Data
(60) Provisional application No. 60/507,385, filed on Sep. 30, 2003.

(51) Int. Cl. A61K 31/4706; C07D 215/38

(52) U.S. Cl. 514/313; 546/159

(57) ABSTRACT

Compounds resulting from the administration of torcetrapib to a mammal, and the use of such compounds as an indicator or biomarker to the presence or exposure of torcetrapib in the plasma of a mammal including humans. The invention is also directed to cholesteryl ester transfer protein (CETP) inhibitors, pharmaceutical compositions containing such inhibitors and the use of such inhibitors to elevate certain plasma lipid levels, including high density lipoprotein (HDL)-cholesterol and to lower certain other plasma lipid levels, such as low density lipoprotein (LDL)-cholesterol and triglycerides.

### BACKGROUND OF THE INVENTION

The invention relates to one or more compounds resulting from the administration of 4-[(3,5-bis-trifluoromethyl-benzyl)-methoxycarbonyl-amino]-2-ethyl)-6-trifluoromethyl-3,4-dihydro-2H-quinoline-1-carboxylic acid ethyl ester, hereafter “torcetrapib”, to a mammal. The compounds can therefore be used as an indicator or biomarker to the presence or exposure of torcetrapib in plasma of a mammal including humans.

This invention also relates to cholesteryl ester transfer protein (CETP) inhibitors, pharmaceutical compositions containing such inhibitors and the use of such inhibitors to elevate certain plasma lipid levels, including high density lipoprotein (HDL)-cholesterol and to lower certain other plasma lipid levels, such as low density lipoprotein (LDL)-cholesterol and triglycerides. Accordingly, the CETP inhibitors can be used to treat diseases which are affected by low levels of HDL-cholesterol and/or high levels of LDL-cholesterol and triglycerides, such as atherosclerosis and cardiovascular diseases in certain mammals, i.e., those mammals that have CETP in their plasma, including humans. Atherosclerosis and its associated coronary artery disease (CAD) is the leading cause of mortality in the industrialized world. Despite attempts to modify secondary risk factors, e.g., smoking, obesity, lack of exercise, and treatment of dyslipidemia with dietary modification and drug therapy, coronary heart disease (CHD) remains the most common cause of death in the U.S. Cardiovascular disease accounts for 44% of all deaths, with 53% of these associated with atherosclerotic coronary heart disease.

Risk for development of this condition has been shown to be strongly correlated with certain plasma lipid levels. Elevated LDL-cholesterol is recognized as a significant contributor to CHD. Low HDL-cholesterol is also a known risk factor for CHD (Gordon, D. J., et al.,: “High-density Lipoprotein Cholesterol and Cardiovascular Disease”, Circulation, (1989), 79: 8-15). High LDL-cholesterol and triglyceride levels are positively correlated, while high levels of HDL-cholesterol are negatively correlated with the risk for developing cardiovascular diseases. Thus, dyslipidemia is not a unitary risk profile for CHD but may be comprised of one or more lipid aberrations.

Among the many factors controlling plasma levels of these disease dependent principles, cholesteryl ester transfer protein (CETP) activity affects all three. The role of this 70,000 dalton plasma glycoprotein found in a number of animal species, including humans, is to transfer cholesteryl ester and triglyceride between lipoprotein particles, including high density lipoproteins, low density lipoproteins, very low density lipoproteins (VLDL), and chylomicrons. The net result of CETP activity is a lowering of HDL-cholesterol and an increase in LDL-cholesterol. This effect on lipoprotein profile is believed to be pro-atherogenic, especially in subjects whose lipid profile constitutes an increased risk for CHD.

EP0818448 (970624) discloses the preparation of certain 5,6,7,8 substituted tetrahydroquinolines and their use as CETP inhibitors. U.S. Pat. No. 5,231,102 discloses a class of 4-substituted 1,2,3,4-tetrahydroquinolines that possess an acidic group (or group convertible thereto in vivo) at the 2-position that are specific antagonists of N-methyl-D-aspartate (NMDA) receptors and are therefore useful in the treatment and/or prevention of neurodegenerative disorders. U.S. Pat. No. 5,288,725 discloses pyrroloquinoline bradykinin antagonists.

Although there are a variety of anti-atherosclerosis therapies, there is a continuing need and a continuing search in this field of art for alternative therapies. U.S. Pat. No. 6,197,786 discloses a class of substituted-3,4-dihydro 2H-quinolines as CETP inhibitors. Of particular interest is torcetrapib, and its use for raising levels of HDL-cholesterol or lowering levels of LDL-cholesterol. Accordingly, there exists a need to monitor the presence or exposure of torcetrapib in the plasma of humans.

### SUMMARY OF THE INVENTION

This invention is directed to a compound of Formula I 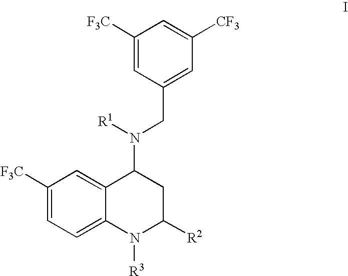
wherein
R¹ is —CO₂CH₃ or —H;
R² is —CH₂CH₃, —CH₂CH₂OH, —CH₂CO₂H, —CH₂CO₂A, and —CH₂CH₂OA, wherein A is 3,4,5-trihydroxy-tetrahydropyran-2-carboxylic acid; and
R³ is —H, —CO₂CH₂CH₃, —CO₂CH₂CH₂OH, —CO₂CH₂CO₂H, —CO₂CH₂CH₂OA and —CO₂CH₂CO₂A; or a pharmaceutically acceptable salt of said compound with the proviso that

if R¹ is —CO₂CH₃ and R³ is —H, then R² is not —CH₂CH₃, —CH₂CH₂OH, and —CH₂CO₂H;

if R¹ is —CO₂CH₃ and R³ is —CO₂CH₂CH₃, then R² is not —CH₂CH₂, CH₂CH₂OH, and —CH₂CO₂H; and

if R¹ is —CO₂CH₃ and R² is —CH₂CH₃, then R³ is not —CO₂CH₂CH₂OH, and —CO₂CH₂CO₂H.

Preferred compounds of Formula I include compounds wherein

R¹ is —CO₂CH₃, R³ is —CO₂CH₂CH₃, and R² is selected from —CH₂CO₂A or —CH₂CH₂OA;

R¹ is —CO₂CH₃, R³ is —H, and R² is selected from —CH₂CO₂A or —CH₂CH₂OA;

R¹ and R³ is H, and R² is selected from —CH₂CH₃, —CH₂CH₂OH, —CH₂CO₂H, —CH₂CO₂A, and —CH₂CH₂OA; and

R¹ is —CO₂CH₃, R² is —CH₂CH₃, and R³ is —CO₂CH₂CO₂A.

The invention is also directed to a compound selected from the following list of compounds. At times each respective compound in this list is referred to herein as a compound-A:
[2R, 4S] 4-[(3,5-bis-trifluoromethyl-benzyl)-methoxycarbonyl-amino]-2-ethyl-6-trifluoromethyl-3,4-dihydro-2H-quinoline-1-carboxylic acid 2-hydroxyethyl ester;
[2R, 4S] 4-[(3,5-bis-trifluoromethyl-benzyl)-methoxycarbonyl-amino]-2-ethyl-6-trifluoromethyl-3,4-dihydro-2H-quinoline-1-carboxylic acid carboxymethyl ester;
[2R, 4S] 4-[(3,5-bis-trifluoromethyl-benzyl)-methoxycarbonyl-amino]-2-carboxymethyl-6-trifluoromethyl-3,4-dihydro-2H-quinoline-1-carboxylic acid ethyl ester;
[2R, 4S] 4-[(3,5-bis-trifluoromethyl-benzyl)-(2-ethyl-6-trifluoromethyl-1,2,3,4-tetrahydro-quinolin-4-yl)-carbamic acid methyl ester;
[2R, 4S] 4-[(3,5-bis-trifluoromethyl-benzyl)-[2-(2-hydroxyethyl)-6-trifluoromethyl-1,2,3,4-tetrahydro-quinolin-4-yl]-carbamic acid methyl ester; and
[2R, 4S] {4-[(3,5-bis-trifluoromethyl-benzyl)-methoxycarbonyl-amino]-6-trifluoromethyl-1,2,3,4-tetrahydro-quinolin-2-yl}-acetic acid

The invention is also directed to a compound of Formula II

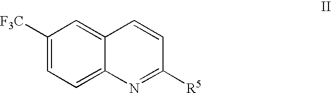

wherein R⁵ is —CH₂CH₃, —CO₂H, —CO₂A, —CH₂CH₂OH, —CH₂CO₂H, —CH₂CH₂OA, —CH₂CH₂OSO₃H, —C(O)N(H)CH₂CH₂SO₃H, —C(O)N(H)CH₂CO₂H, and —C(O)N(H)C(O)NH₂, wherein A is 3,4,5-trihydroxy-tetrahydropyran-2-carboxylic acid.

Preferred compounds of Formula II include the compounds wherein R⁵ is selected from —CH₂CH₃ or —CO₂H.

The invention is also directed to a compound of Formula III

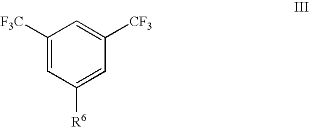

wherein R⁶ is —CH₂OA, —C(O)N(H)CH₂CO₂A and —CH(SO₃H)N(H)CO₂CH₃, wherein A is 3,4,5-trihydroxy-tetrahydropyran-2-carboxylic acid.

The invention is also directed to a method for indicating the presence or exposure of 4-[(3,5-bis-trifluoromethyl-benzyl)-methoxycarbonyl-amino]-2-ethyl)-6-trifluoromethyl-3,4-dihydro-2H-quinoline-1-carboxylic acid ethyl ester, i.e., torcetrapib, in plasma of a mammal including humans by the identification or monitoring of one or more compounds selected from the compounds of Formula I, Formula II, Formula III, a compound-A, or 4-[(3,5-bis-trifluoromethyl-benzyl)-methoxycarbonyl-amino]-(2-hydroxy-ethyl)-6-trifluoromethyl-3,4-dihydro-2H-quinoline-1-carboxylic acid ethyl ester in the mammal. As a result, these compounds can be used as an indicator or biomarker to the presence or exposure of torcetrapib in plasma of a mammal.

One method of the invention includes indicating the presence or exposure of torcetrapib in a mammal by identifying or monitoring a compound of Formula I in the mammal.

Another method of the invention includes indicating the presence or exposure of torcetrapib in a mammal by identifying or monitoring a compound-A or 4-[(3,5-bis-trifluoromethyl-benzyl)-methoxycarbonyl-amino]-(2-hydroxy-ethyl)-6-trifluoromethyl-3,4-dihydro-2H-quinoline-1-carboxylic acid ethyl ester in the mammal.

Another method of the invention includes indicating the presence or exposure of torcetrapib in a mammal by identifying or monitoring a compound of Formula II, 2-methyl-6-trifluoromethyl-quinoline, or (6-trifluoromethyl-quinolin-2-yl)methanol in the mammal.

Another method of the invention includes indicating the presence or exposure of torcetrapib in a mammal by identifying or monitoring a compound selected from

3,5-Bis-trifluoromethyl-benzoic acid, 6-(3,5-Bis-trifluoromethyl-benzoyloxy)-3,4,5-trihydroxy-tetrahydro-pyran-2-carboxylic acid, 6-(3,5-Bis-trifluoromethyl-benzyloxy)-3,4,5-trihydroxy-tetrahydro-pyran-2-carboxylic acid, (3,5-Bis-trifluoromethyl-phenyl)-methoxycarbonylamino-methanesulfonic acid, (3,5-Bis-trifluoromethyl-benzoylamino)-acetic acid, or (3,5-Bis-trifluoromethyl-benzoylamino)-3,4,5-trihydroxy-tetrahydro-pyran-2-carboxylic acid in the mammal.

Another method of the invention includes indicating the presence or exposure of torcetrapib in a mammal by identifying or monitoring a compound selected from

3,5-bis-trifluoromethylbenzoic acid, 2-methyl-6-trifluoromethyl-quinoline, and 6-trifluoromethyl-quinoline-2-carboxylic acid in the mammal.

The invention is also directed to a method for treating atherosclerosis in a mammal comprising administering to a mammal an atherosclerosis treating amount of a compound selected from Formula I, a prodrug thereof, or a pharmaceutically acceptable amount salt of said compound or of said prodrug. The compounds of Formula I are inhibitors of CETP.

The invention is also directed to a method for treating atherosclerosis in a mammal comprising administering to a mammal an atherosclerosis treating amount of a compound-A, a prodrug thereof, or a pharmaceutically acceptable amount salt of said compound or of said prodrug. Any one compound-A can be used to inhibit CETP.

A preferred dosage is about 0.001 to 100 mg/kg/day of a compound of Formula I or of compound-A, a prodrug thereof, or a pharmaceutically acceptable salt of said compound of Formula I or of compound-A or of said prodrug. An especially preferred dosage is about 0.01 to 10 mg/kg/day of a compound of Formula I or of compound-A, a prodrug thereof, or a pharmaceutically acceptable salt of said compound of Formula I or of compound-A or of said prodrug.

The term “treating”, “treat” or “treatment” as used herein includes preventative (e.g., prophylactic) and palliative treatment.

By “pharmaceutically acceptable” is meant the carrier, diluent, excipients, and/or salt must be compatible with the other ingredients of the formulation, and not deleterious to the recipient thereof.

The expression “prodrug” refers to compounds that are drug precursors which following administration, release the drug in vivo via some chemical or physiological process (e.g., a prodrug on being brought to the physiological pH or through enzyme action is converted to the desired drug form). Exemplary prodrugs upon cleavage release the corresponding free acid, and such hydrolyzable ester-forming residues of the compounds of Formula I and of a compound-a.

The expression “pharmaceutically-acceptable salt” refers to nontoxic anionic salts containing anions such as (but not limited to) chloride, bromide, iodide, sulfate, bisulfate, phosphate, acetate, maleate, fumarate, oxalate, lactate, tartrate, citrate, gluconate, methanesulfonate and 4-toluene-sulfonate. The expression also refers to nontoxic cationic salts such as (but not limited to) sodium, potassium, calcium, magnesium, ammonium or protonated benzathine (N,N′-dibenzylethylenediamine), choline, ethanolamine, diethanolamine, ethylenediamine, meglamine (N-methylglucamine), benethamine (N-benzylphenethylamine), piperazine or tromethamine (2-amino-2-hydroxymethyl-1,3-propanediol).

The chemist of ordinary skill will recognize that certain compounds of this invention will contain one or more atoms which may be in a particular stereochemical or geometric configuration, giving rise to stereoisomers and configurational isomers. All such isomers and mixtures thereof are included in this invention. Hydrates and solvates of the compounds of this invention are also included.

### DETAILED DESCRIPTION OF THE INVENTION

The compounds of Formula I, Formula II and Formula III are metabolites of torcetrapib. As a result, these compounds (metabolites) can be used as biomarkers to the presence or exposure of torcetrapib in the plasma of mammals including humans by identifying or monitoring the presence of one or more compounds selected from compounds of Formula I, Formula II or Formula III in the mammal. The compounds of Formula I, Formula II and Formula III can be isolated from the plasma, feces or urine of mammals following administration, preferably oral administration, of torcetrapib to the mammals including humans. Consequently, the compounds of Formula I, Formula II and Formula III can be prepared by administering torcetrapib to a human or other mammal and isolating the desired compound (metabolite) from plasma, urine, or feces from the human subject or mammal. The compounds of Formula I, Formula II and Formula III can also be synthetically prepared using methods described in this Application as well as alternative synthetic methods known to those of ordinary skill in the art. As a result, the metabolites of torcetrapib isolated from the administration of torcetrapib to a mammal can be structurally verified by comparison with the HPLC and/or mass spectroscopic data of the corresponding synthetically prepared compounds.

The following list compounds of the invention, referred to herein as a compound-A, are also metabolites of torcetrapib:
[2R, 4S] 4-[(3,5-bis-trifluoromethyl-benzyl)-methoxycarbonyl-amino]-2-ethyl-6-trifluoromethyl-3,4-dihydro-2H-quinoline-1-carboxylic acid 2-hydroxyethyl ester;
[2R, 4S] 4-[(3,5-bis-trifluoromethyl-benzyl)-methoxycarbonyl-amino]-2-ethyl-6-trifluoromethyl-3,4-dihydro-2H-quinoline-1-carboxylic acid carboxymethyl ester;
[2R, 4S] 4-[(3,5-bis-trifluoromethyl-benzyl)-methoxycarbonyl-amino]-2-carboxymethyl-6-trifluoromethyl-3,4-dihydro-2H-quinoline-1-carboxylic acid ethyl ester;
[2R, 4S] 4-[(3,5-bis-trifluoromethyl-benzyl)-(2-ethyl-6-trifluoromethyl-1,2,3,4-tetrahydro-quinolin-4-yl)-carbamic acid methyl ester;
[2R, 4S] 4-[(3,5-bis-trifluoromethyl-benzyl)-[2-(2-hydroxyethyl)-6-trifluoromethyl-1,2,3,4-tetrahydro-quinolin-4-yl]-carbamic acid methyl ester; and
[2R, 4S] {4-[(3,5-bis-trifluoromethyl-benzyl)-methoxycarbonyl-amino]-6-trifluoromethyl-1,2,3,4-tetrahydro-quinolin-2-yl}-acetic acid.

As a result, any one compound-A can be used as a biomarker to the presence or exposure of torcetrapib in the plasma of mammals including humans by identifying or monitoring the presence of the compound in the mammal. Any one compound-A can be isolated from the plasma, feces, or urine of mammals following administration, preferably oral administration, of torcetrapib to the mammals including humans. Consequently, any one compound-A can be prepared by administering torcetrapib to a human or other mammal and isolating the compound from plasma, urine, or feces from the human subject or mammal. Any one compound-A can also be synthetically prepared using methods described in this Application as well as alternative synthetic methods known to those of ordinary skill in the art. As a result, these specific metabolites of torcetrapib isolated from the administration of torcetrapib to a mammal can be structurally verified by comparison with the HPLC and/or mass spectroscopic data of the synthetically prepared compounds.

The compounds 3,5-Bis-trifluoromethyl-benzoic acid, 6-(3,5-Bis-trifluoromethyl-benzoyloxy)-3,4,5-trihydroxy-tetrahydro-pyran-2-carboxylic acid, 6-(3,5-Bis-trifluoromethyl-benzyloxy)-3,4,5-trihydroxy-tetrahydro-pyran-2-carboxylic acid, (3,5-Bis-trifluoromethyl-phenyl)-methoxycarbonylamino-methanesulfonic acid, (3,5-Bis-trifluoromethyl-benzoylamino)-acetic acid, 2-methyl-6-trifluoromethyl-quinoline, (6-trifluoromethyl-quinolin-2-yl)methanol, or (3,5-Bis-trifluoromethyl-benzoylamino)-3,4,5-trihydroxy-tetrahydro-pyran-2-carboxylic acid are also metabolites of torcetrapib. These compounds can be isolated from the plasma, feces, or urine of mammals following administration, preferably oral administration, of torcetrapib to the mammals including humans. Consequently, the compounds can be prepared by administering torcetrapib to a human or other mammal and isolating the compound from plasma, urine, or feces from the human subject or mammal.

As a result, any one compound selected from 3,5-Bis-trifluoromethyl-benzoic acid, 6-(3,5-Bis-trifluoromethyl-benzoyloxy)-3,4,5-trihydroxy-tetrahydro-pyran-2-carboxylic acid, 6-(3,5-Bis-trifluoromethyl-benzyloxy)-3,4,5-trihydroxy-tetrahydro-pyran-2-carboxylic acid, (3,5-Bis-trifluoromethyl-phenyl)-methoxycarbonylamino-methanesulfonic acid, (3,5-Bis-trifluoromethyl-benzoylamino)-acetic acid, 2-methyl-6-trifluoromethyl-quinoline, (6-trifluoromethyl-quinolin-2-yl)methanol, or (3,5-Bis-trifluoromethyl-benzoylamino)-3,4,5-trihydroxy-tetrahydro-pyran-2-carboxylic acid can be used as a biomarker to the presence or exposure of torcetrapib in the plasma of mammals including humans by identifying or monitoring the presence of at least one of the listed compounds in the mammal.

As an initial note, some of the preparation methods used in the preparation of the compounds of the invention may require protection of remote functionality (e.g., primary amine, secondary amine, carboxyl, or hydroxyl). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see T. W. Greene, Protective Groups in Organic Synthesis, John Wiley & Sons, New York, 1991.

For example, in reaction Schemes 1 to 4 certain compounds contain primary amines or carboxylic acid functionalities which may interfere with reactions at other sites of the molecule if left unprotected. Accordingly, such functionalities may be protected by an appropriate protecting group which may be removed in a subsequent step. Suitable protecting groups for amine and carboxylic acid protection include those protecting groups commonly used in peptide synthesis (such as N-t-butoxycarbonyl, benzyloxycarbonyl, and 9-fluorenylmethylenoxycarbonyl for amines and lower alkyl or benzyl esters for carboxylic acids) which are generally not chemically reactive under the reaction conditions described and can typically be removed without chemically altering other functionality in the compound.

In general the compounds of this invention can be made by processes which include processes analogous to those known in the chemical arts, particularly in light of the description contained herein. Certain processes for the manufacture of the compounds of this invention are provided as further features of the invention and are illustrated by reaction Schemes 1 to 4. Detailed synthetic procedures used to prepare one or more compounds of Formula I, Formula II, Formula III and of a compound-A are described in the Example section of this Application.

The compounds of Formula I and of compound-A can be prepared according to the synthetic procedures described in U.S. Pat. No. 6,197,786, and U.S. patent application Ser. No. 10/137,314, the entire disclosures of which are incorporated herein by reference.

In particular, the tetrahydroquinoline ring system is prepared by treating the appropriate aromatic amine with the requisite carboxaldehyde in an inert solvent such as a hydrocarbon (e.g., hexanes, pentanes or cyclohexane), an aromatic hydrocarbon, a halocarbon, an ether, a nitrile, a nitroalkane, preferably dichloromethane with a dehydrating agent (e.g., sodium sulfate or magnesium sulfate) at a temperature of about 0° C. to about 100° C. (preferably ambient temperature) for 1 to 24 hours (preferably 1 hour). The resulting solution is treated with a suitably substituted (e.g., benzyloxycarbonyl, t-butoxycarbonyl, methoxycarbonyl, formyl-, acetyl-, diallyl- or dibenzyl-), preferably carboxybenzyloxy-, N-vinyl species and with a Lewis acid (e.g., boron trifluoride, boron trifluoride etherate, zinc chloride, titanium tetrachloride, iron trichloride, aluminum trichloride, alkyl aluminum dichloride, dialkyl aluminum chloride or ytterbium (III) triflate; preferably boron trifluoride etherate) or a protic acid at a temperature of from about −78° C. to about 50° C. (preferably ambient temperature) for 0.1 to 24 hours (preferably 1 hour).

The resulting 6-trifluoromethyl quinoline ring system ring with the appropriate R² substituent is then reacted with 3,5-bis-trifluoromethyl benzaldehyde in a reductive condensation reaction as indicated in-part in Scheme 1.

Alternatively, the aromatic amine and appropriate carboxaldehyde may be condensed in the presence of 1H-benzotriazole by combining the three components in a suitable solvent (preferably toluene) as shown in-part in Scheme 1. The reaction is typically conducted at a temperature between 0 and reflux (preferably about ambient temperature) for between 15 minutes and 24 hours (preferably about 2 hours). The reaction apparatus is optionally equipped for the azeotropic removal of water. After the reaction is at or near completion, as indicated by TLC, GC, NMR or other means, the reaction mixture is concentrated to afford the imine-benzotriazole adduct. The residue is slurried in a nonpolar solvent (preferably hexanes) and the resulting suspension collected by filtration. The resulting imine-benzotriazole adduct can then be used to prepare the desired tetrahydroquinoline system of Formula 1 or Compound-A.

The compounds of Formula I and of compound-A in which R² is not —CH₂CH₃ can be prepared from Intermediate A, Intermediate B, or Intermediate C, whose multi-step synthesis is summarized in Scheme 1. A ring-closing step is used to form the

6-trifluoromethyl quinoline ring system followed by a reductive condensation reaction with 3,5-bis-trifluoromethyl benzaldehyde. Standard chemical conversions, such as those described in U.S. Pat. No. 6,197,786 and in the preparation of glucoronidated products, can be used to convert these intermediates to the desired compounds of Formula I and compound-A.

For example, compound 19 can be prepared from Intermediate C in three basic steps: (1) hydrogenation over Pd/C providing compound 24; (2) protection of the alcohol; and (3) esterfication of the quinoline-nitrogen. Compounds 21 and 26 can be prepared from compounds 19 and 24, respectively, with known procedures to convert an alcohol into a carboxylic acid.

The glycoside compounds 20 and 25 can be prepared from compounds 19 and 24, respectively, by the reaction of the alcohol and carbohydrate in an inert solvent such as toluene. An alternative procedure is the reaction of the alcohol with a protected glycosyl halide in the presence of base followed by deprotection. Similarly, glycoside compounds 22, 27 and 32 can be prepared from compounds 21, 26 and 31, respectively, by the reaction of the acid and carbohydrate. Finally, the glycoside compounds can be prepared according to the in-vitro enzymatic procedure described in the Example section of this Application.

The 2-substituted-6-trifluoromethyl-quinoline compounds of Formula II can be prepared in accordance with Schemes 2, 3 or 4. The compound numbers identified in Schemes 2, 3 and 4 correspond to the compound numbers provided in Tables 2 and 3. The 2-(6-trifluormethyl)-quinolin-2yl)-amide compounds 12, 13 and 14 can be prepared from the corresponding acid, compound 11, using synthetic procedures well known to those of ordinary skill in the art.

The 1,3,5-trisubstituted phenyl compounds of Formula III can be prepared starting from 3,5-bis-trifluoromethyl benzaldehyde using synthetic procedures well known to those of ordinary skill in the art. 3,5-bis-trifluoromethylbenzoic acid can be prepared according to the procedure described in U.S. Pat. No. 6,489,507, of which the entire disclosure is incorporated herein by reference.

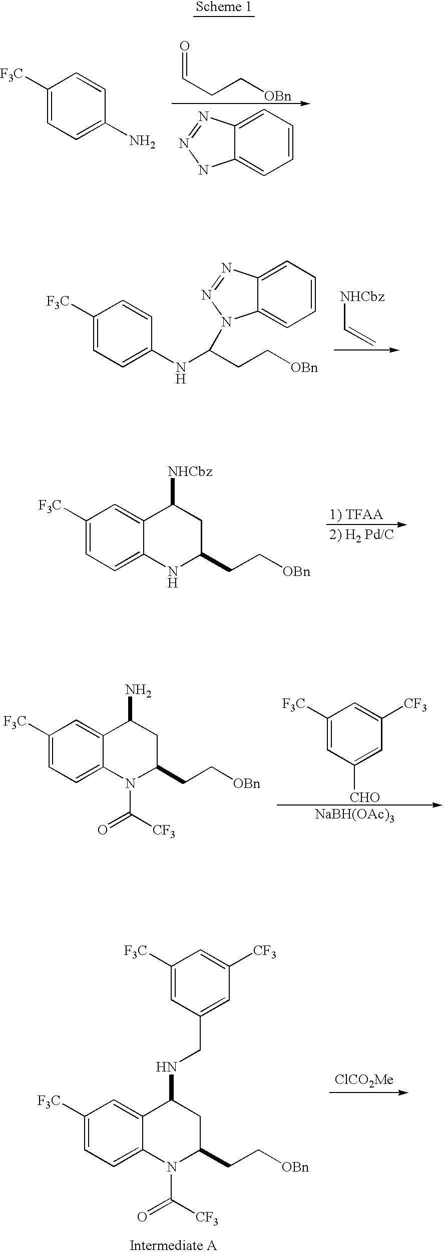

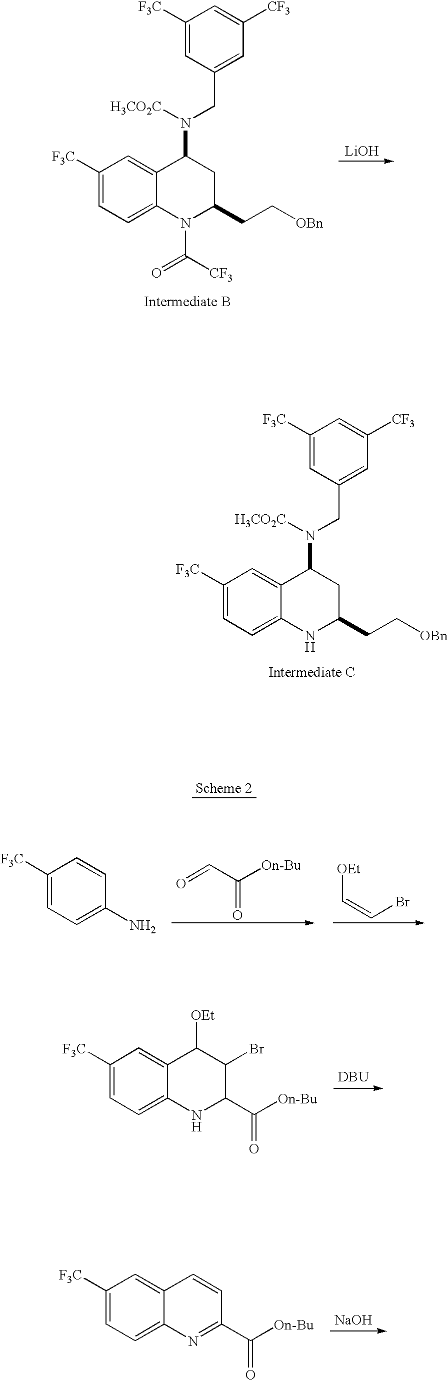

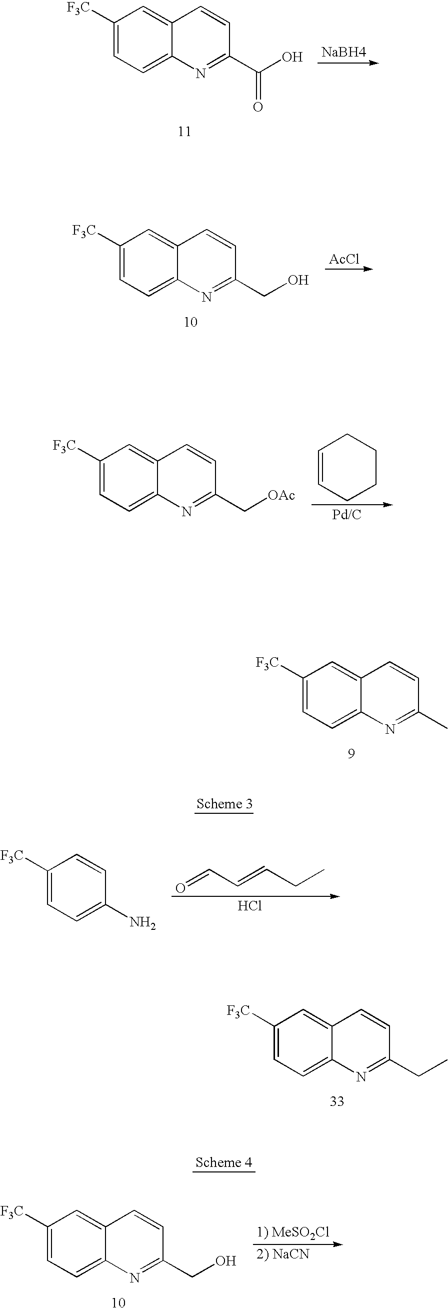

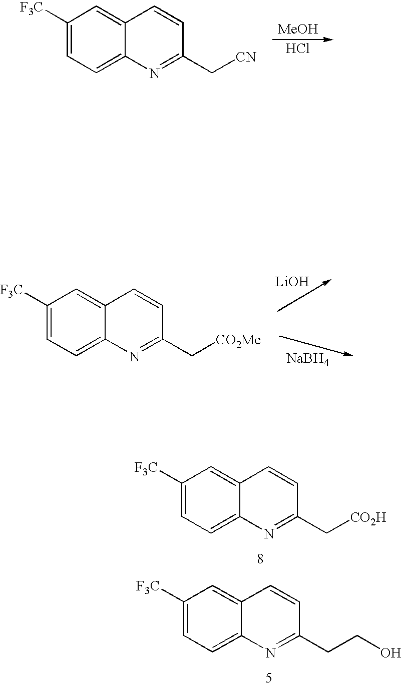

Prodrugs of compounds of Formula I and of any one compound-A can be prepared according to methods known to those skilled in the art. For example, a carboxyl group in a carboxylic acid of a compound of Formula I or a compound-A can be replaced by an ester prepared by combining the carboxylic acid with the appropriate alkyl halide in the presence of a base such as potassium carbonate in an inert solvent. Alternatively, an alcohol function can be derivatized as an ether prepared by combining the alcohol with the appropriate alkyl bromide or iodide in the presence of a base such as potassium carbonate in an inert solvent.

The starting materials and reagents for compounds of Formula I, Formula II, Formula III and of any one compound-A are readily available or can be easily synthesized by those skilled in the art using conventional methods of organic synthesis. For example, many of the compounds used herein, are related to, or are derived from compounds in which there is a large scientific interest and commercial need, and accordingly many such compounds are commercially available or are reported in the literature or are easily prepared from other commonly available substances by methods which are reported in the literature.

Some of the compounds of Formula I, Formula II, Formula III, and of a compound-A, or intermediates in their synthesis have asymmetric carbon atoms and therefore are enantiomers or diastereomers. Diastereomeric mixtures can be separated into their individual diastereomers on the basis of their physical chemical differences by methods known per, example, by chromatography and/or fractional crystallization. Enantiomers can be separated by, for example, chiral HPLC methods or converting the enantiomeric mixture into a diastereomeric mixture by reaction with an appropriate optically active compound (e.g., alcohol), separating the diastereomers and converting (e.g., hydrolyzing) the individual diastereomers to the corresponding pure enantiomers. Also, a racemic mixture of the compounds of Formula I, Formula II, Formula III or of a compound-A, or an intermediate in their synthesis which contain an acidic or basic moiety may be separated into their compounding pure enantiomers by forming a diastereomeric salt with an optically pure chiral base or acid (e.g., 1-phenyl-ethyl amine or tartaric acid) and separating the diastereomers by fractional crystallization followed by neutralization to break the salt, thus providing the corresponding pure enantiomers. All such isomers, including diastereomers, enantiomers and mixtures thereof are considered as part of this invention.

More specifically, the enantiomeric compounds of Formula I, Formula II, Formula III and of a compound-A can be obtained in enantiomerically enriched form by resolving the racemate of the final compound or an intermediate in its synthesis (preferably the final compound) employing chromatography (preferably high pressure liquid chromatography [HPLC]) on an asymmetric resin (preferably Chiralcel™ AD or OD [obtained from Chiral Technologies, Exton, Pa.]) with a mobile phase consisting of a hydrocarbon (preferably heptane or hexane) containing between 0 and 50% isopropanol and between 0 and 5% of an alkyl amine. Concentration of the product containing fractions affords the desired materials.

Some of the compounds of Formula I, Formula II, Formula II and of a compound-A are acidic and they form a salt with a pharmaceutically acceptable cation. Likewise, some of the compounds of Formula I, Formula II, Formula III and of a compound-A are basic and they form a salt with a pharmaceutically acceptable anion. All such salts are within the scope of this invention and they can be prepared by conventional methods such as combining the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate. The compounds can be obtained in crystalline form by dissolution in an appropriate solvent(s) such as ethanol, hexanes -an or water/ethanol mixtures. In addition, if a compound of Formula I, Formula II, Formula III or of a compound-A form hydrates or solvates, the hydrates and solvates are also within the scope of the invention.

The compounds of Formula 1 and any one of compound-A and the salts of such compounds can be adapted to therapeutic use as agents that inhibit CETP activity in mammals, particularly humans or can be used as an indicator of the active CETP inhibitor in their plasma. These compounds elevate plasma HDL cholesterol, its associated components, and the functions performed by them in mammals, particularly humans. By virtue of their activity, these agents also reduce plasma levels of triglycerides, VLDL cholesterol LDL-cholesterol and their associated components in mammals, particularly humans. Hence, these compounds are useful for the treatment and correction of the various dyslipidemias observed to be associated with the development and incidence of atherosclerosis and cardiovascular disease, including hypoalphalipoproteinemia, hyperbetalipoproteinemia, hypertriglyceridemia, and familial-hypercholesterolemia.

Given the negative correlation between the levels of HDL cholesterol and HDL associated lipoproteins, and the positive correlation between triglycerides, LDL-cholesterol, and their associated apolipoproteins in blood with the development of cardiovascular, cerebral vascular and peripheral vascular diseases, compounds of Formula I compounds or of any one compound-A and the salts of such compounds, by virtue of their pharmacologic action, are useful for the prevention, arrestment and/or regression of atherosclerosis and its associated disease states. These include cardiovascular disorders (e.g., angina, cardiac ischemia and myocardial infarction), complications due to cardiovascular disease therapies (e.g., reperfusion injury and angioplastic restenosis), hypertension, stroke, and atherosclerosis associated with organ transplantation.

Because of the beneficial effects widely associated with elevated HDL levels, an agent which inhibits CETP activity in humans, by virtue of its HDL increasing ability, also provides valuable avenues for therapy in a number of other disease areas as well.

The utility of compounds of Formula I or of any one compound-A, their prodrugs and the salts of such compounds and prodrugs as medical agents in the treatment of the above described disease/conditions in mammals (e.g. humans, male or female) is demonstrated by the activity of the compounds of this invention in conventional assays and the in vivo assay described below. The in vivo assay (with appropriate modifications within the skill in the art) may be used to determine the activity of other lipid or triglyceride controlling agents as well as the compounds of this invention. Such assays also provide a means whereby the activities of the compounds and the salts of such compounds (or the other agents described herein) can be compared to each other and with the activities of other known compounds. The results of these comparisons are useful for determining dosage levels in mammals, including humans, for the treatment of such diseases.

The hyperalphacholesterolemic activity of compounds of Formula I or of any one compound-A can be determined by assessing the effect of these compounds on the action of cholesteryl ester transfer protein by measuring the relative transfer ratio of radiolabeled lipids between lipoprotein fractions, essentially as previously described by Morton in J. Biol. Chem. 1981 256, 11992, and by Dias in Clin. Chem 1988, 34, 2322, 1988.

1. CETP In-vitro Assay.

The following is a brief description of the assay of cholesteryl ester transfer in human plasma (in vitro) and animal plasma (ex vivo): CETP activity in the presence or absence of drug is assayed by determining the transfer of 3H— labeled cholesteryl oleate (CO) from exogenous tracer HDL to the non-HDL lipoprotein fraction in human plasma, or from 3H-labeled LDL to the HDL fraction in transgenic mouse plasma. Labeled human lipoprotein substrates are prepared similarly to the method described by Morton in which the endogenous CETP activity in plasma is employed to transfer 3H—CO from phospholipid liposomes to all the lipoprotein fractions in plasma. 3H-labeled LDL and HDL are subsequently isolated by sequential ultracentrifugation at the density cuts of 1.019-1.063 and 1.10-1.21 g/ml, respectively. For the activity assay, 3H— labeled lipoprotein is added to plasma at 10-25 nmoles CO/ml and the samples incubated at 37° C. for 2.5-3 hrs. Non-HDL lipoproteins are then precipitated by the addition of an equal volume of 20% (wt/vol) polyethylene glycol 8000 (Dias). The samples are centrifuged 750 g×20 minutes and the radioactivity contained in the HDL containing supernatant determined by liquid scintillation. Introducing varying quantities of the compounds of this invention as a solution in dimethylsulfoxide to human plasma, before addition of the radiolabeled cholesteryl oleate, and comparing the relative amounts of radiolabel transferred allows relative cholesteryl ester transfer inhibitory activities to be determined.

2. Plasma Lipids Assay.

The activity of these compounds may also be demonstrated by determining the amount of agent required to alter plasma lipid levels, for example HDL cholesterol levels, LDL cholesterol levels, VLDL cholesterol levels or triglycerides, in the plasma of certain mammals, for example marmosets that possess CETP activity and a plasma lipoprotein profile similar to that of humans (Crook et al. Arteriosclerosis 1990 10, 625.). Adult marmosets are assigned to treatment groups so that each group has a similar mean+−SD for total, HDL, and/or LDL plasma cholesterol concentrations. After group assignment, marmosets are dosed daily with compound as a dietary admix or by intragastric intubation for from one to eight days. Control marmosets receive only the dosing vehicle. Plasma total, LDL, VLDL and HDL cholesterol values can be determined at any point during the study by obtaining blood from an antecubital vein and separating plasma lipoproteins into their individual subclasses by density gradient centrifugation, and by measuring cholesterol concentration as previously described by Crook et al.

3. In-vivo Atherosclerosis Assay.

Anti-atherosclerotic effects of the compounds can be determined by the amount of compound required to reduce the lipid deposition in rabbit aorta. Male New Zealand White rabbits are fed a diet containing 0.2% cholesterol and 10% coconut oil for 4 days (meal-fed once per day). Rabbits are bled from the marginal ear vein and total plasma cholesterol values are determined from these samples. The rabbits are then assigned to treatment groups so that each group has a similar mean +−SD for total plasma cholesterol concentration, HDL cholesterol concentration, triglyceride concentration and/or cholesteryl ester transfer protein activity. After group assignment, rabbits are dosed daily with compound given as a dietary admix or on a small piece of gelatin based confection. Control rabbits receive only the dosing vehicle, be it the food or the gelatin confection. The cholesterol/coconut oil diet is continued along with the compound administration throughout the study. Plasma cholesterol values and cholesteryl ester transfer protein activity can be determined at any point during the study by obtaining blood from the marginal ear vein. After 3-5 months, the rabbits are sacrificed and the aortae are removed from the thoracic arch to the branch of the iliac arteries. The aortae are cleaned of adventitia, opened longitudinally and then stained with Sudan IV as described by Holman et. al. (Lab. Invest. 1958, 7, 42-47). The percent of the surface area stained is quantitated by densitometry using an Optimas Image Analyzing System (Image Processing Systems). Reduced lipid deposition is indicated by a reduction in the percent surface area stained in the compound-receiving group in comparison with the control rabbits.

Administration of the compounds of Formula I or of any one compound-A can be administered via any method which delivers a compound of this invention systemically and/or locally. These methods include oral routes, parenteral, intraduodenal routes, etc. Generally, the compounds of this invention are administered orally, but parenteral administration (e.g., intravenous, intramuscular, subcutaneous or intramedullary) may be utilized, for example, where oral administration is inappropriate for the target or where the patient is unable to ingest the drug.

In general an amount of an active CETP inhibitor is used that is sufficient to achieve the therapeutic effect desired (e.g., HDL elevation). An effective dosage for the CETP inhibitors of this invention, their prodrugs and the salts of such compounds and prodrugs is in the range of 0.01 to 10 mg/kg/day, preferably 0.1 to 5 mg/kg/day.

The CETP inhibitors of this invention are generally administered in the form of a pharmaceutical composition comprising at least one of the compounds together with a pharmaceutically acceptable vehicle, diluent or carrier. Thus, the CETP inhibitors can be administered individually or together in any conventional oral, parenteral, rectal or transdermal dosage form.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. A preferred formulation is a solution or suspension in an oil, for example olive oil, Miglyol™ or Capmul™, in a soft gelatin capsule. Antioxidants may be added to prevent long term degradation as appropriate. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easter, Pa., 15th Edition (1975).

Pharmaceutical compositions according to the invention may contain 0.1%-95% of the compound(s) of this invention, preferably 1%-70%. In any event, the composition or formulation to be administered will contain a quantity of a compound(s) according to the invention in an amount effective to treat the disease/condition of the subject being treated, e.g., atherosclerosis.

Example Section

1. Sample analysis for determining the metabolic profile of torcetrapib and the identification and preparation of the metabolites using HPLC/MS.

All mammals including human subjects were dosed with [¹⁴C] 4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester, that is, ¹⁴C-labled torcetrapib. The urine, feces and plasma of the mammals and human subjects was collected for metabolite profiling.

Feces: Fecal homogenates were pooled such that 90% or greater radioactivity was accounted for. Each pooled fecal sample was diluted with 30 mL acetonitrile and vortexed. The sample was centrifuged and the supernatant evaporated to about 1 mL in a turbovap at 35° C. under nitrogen. The process was repeated several times until>90% of the radioactivity was extracted. After adding 10 mL of ethyl acetate into the tube for liquid-liquid extraction, the ethyl acetate layer was removed and evaporated in a turbovap at 35° C. under nitrogen. The process was repeated several times until>90% of the radioactivity is extracted. The residue obtained was reconstituted in ˜0.3 mL of acetonitrile:H₂O (3:1). An aliquot (about 100 μl) of the reconstituted sample was then injected into the HPLC column to separate and structurally identify the metabolites.

The percentage of metabolites in fecal extract was determined by measuring the radioactivity in the individual peaks that are separated on HPLC using β-RAM detector (IN/US, Win-flow). The β-RAM was operated in the homogeneous liquid scintillation counting mode with the addition of 3 ml/min of Tru-Count scintillation cocktail (IN/US) to the effluent post-UV detection.

Plasma: Plasma samples were pooled according to the method reported by Hamilton and co-workers, for profiling of circulating metabolites (Hamilton R. A. et. al. 1981). The pooled plasma samples were treated with acetonitrile (5-fold excess). The mixture was centrifuged and the supernatant evaporated to about 2 mL in a turbovap at 35° C. under nitrogen. The concentrated samples were loaded onto Isolute C18 SPE columns (500 mg) and the columns subsequently washed with acetonitrile. The washing was continued so that >90% of the radioactivity was recovered from SPE columns. Both aqueous and acetonitrile fractions were evaporated to dryness. The residue from the aqueous fraction was dissolved in acetonitrile and centrifuged. The supernatant was mixed with the acetonitrile fraction and evaporated to dryness. The final residue was reconstituted in ˜300 μl of 2:1 acetonitrile:water. An aliquot (about 100 μl) of the reconstituted sample was then injected into the HPLC column to separate and structurally identify the metabolites.

Urine: Urine was pooled such that greater than 90% of the excreted radioactivity was accounted for. The pooling was proportional to the volumes of urine collected at each time point. Pooled urine samples were precipitated with five fold the volume of acetonitrile and then centrifuged (3000 rpm for 10 min). The supernatants were evaporated in a turbovap at 35° C. under nitrogen. The residue obtained was reconstituted in about 0.5 mL of 1:1 acetonitrile:H₂O. An aliquot (about 100 μl) of the reconstituted sample was then injected into the HPLC column to separate and structurally identify the metabolites.

High Performance Liquid Chromatography: The HPLC system consisted of a HP-1100 solvent delivery pump, a HP-1100 membrane degasser, a HP-1100 autoinjector and an IN/US radioactive monitor (β-RAM). Chromatography was performed on a Zorbax C18 column (5 micron, 4.5×150 mm) by injecting 100 μl of the reconstituted sample. The mobile phase was initially composed of acetonitrile (solvent A) and 10 mM ammonium formate (pH 2.0) (solvent B). The flow rate was 1.0 mL/min and separation was achieved at ambient temperature. The 60 minute gradient is summarized in Table 1.

TABLE 1

| | % Solvent A | % Solvent B |
|---|---|---|
| Time (min) | (Acetonitrile) | (10 mM, Ammonium formate) |
| 0-10 | 20 | 80 |
| 10-35 | 24 | 76 |
| 35-37 | 50 | 50 |
| 37-53 | 95 | 5.0 |
| 53-55 | 20 | 80 |

The system was allowed to equilibrate for 5 min prior to the next injection. The post-column eluate was split such that 95% of the flow was monitored continuously with a β-RAM online detector fitted with a liquid scintillation cell (IN/US). The remaining 5% of the flow was diverted to a PE SCIEX API 2000 mass spectrometer or Finnigan LCQ Ion Trap mass spectrometer. The peaks in the radiochromatogram were quantified using Winflow software (INUS, Riviera Beach, Fla.) or LC-ARC software (AIM Research Company, DE) by measuring the radioactivity in the individual peaks separated on HPLC using β-RAM or LC-ARC. The radioactivity response was also recorded in real time by the mass spectrometer data system. This allowed simultaneous real time monitoring of radioactivity and the detection of the total ion chromatogram.

All mass spectrometers were, unless mentioned otherwise, operated in the positive ion mode. Data was collected in the Q1 scanning, neutral loss scanning, precursor ion scanning, product ion scanning, multiple reaction monitoring scanning and data-dependent ion scanning modes, with instrument settings and potentials (e.g., collision energy) adjusted to provide optimal data in each mode.

The metabolites in urine and plasma were quantified by measuring the individually separated radioactive peaks using LC-ARC system (Liquid Chromatography-Accurate Radioisotope Counting, AIM Research Company). The LC-ARC was operated in the homogeneous liquid scintillation counting mode with the addition of 2.5 mL/min of Tru-Count scintillation cocktail (IN/US) to the effluent post-UV detection. The percentage of metabolite M1 (BTFMBA) excreted in the urine was determined by quantifying the concentrations of BTFMBA in urine samples at each sampling time.

Table 2 lists the metabolites of torcetrapib separated and identified by HPLC and mass spectroscopy, respectively. Table 2 also lists the HPLC retention time recorded for each metabolite identified by compound number in the third column of Table 2 using the separation conditions described above.

TABLE 2

| | | | Retention |
|---|---|---|---|
| | | | Time |
| Compound Structure | Compound Name | No. | min |
| 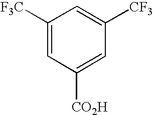 | 3,5-Bis-trifluoromethyl- benzoic acid | 1 | 27 |
| 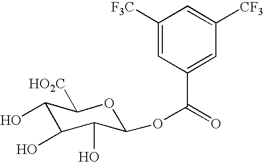 | 6-(3,5-Bis-trifluoromethyl- benzoyloxy)-3,4,5- trihydroxy-tetrahydro- pyran-2-carboxylic acid | 2 | 15 |
| 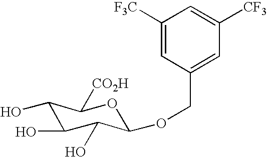 | 6-(3,5-Bis-trifluoromethyl- benzyloxy)-3,4,5- trihydroxy-tetrahydro- pyran-2-carboxylic acid | 3 | 15 |
| 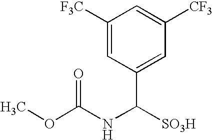 | (3,5-Bis-trifluoromethyl- phenyl)- methoxycarbonylamino- methanesulfonic acid | 4 | 15-16 |
| 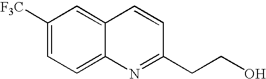 | 2-(6-Trifluoromethyl- quinolin-2-yl)-ethanol | 5 | 18 |
| 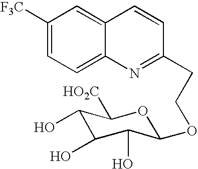 | 3,4,5-Trihydroxy-6-[2-(6- trifluoromethyl-quinolin-2- yl)-ethoxy]-tetrahydro- pyran-2-carboxylic acid | 6 | 16 |
| 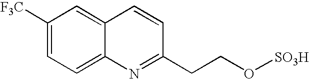 | Sulfuric acid mono-[2-(6- trifluoromethyl-quinolin-2- yl)-ethyl] ester | 7 | 18-19 |
| 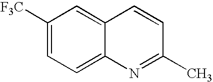 | 2-Methyl-6-trifluoromethyl- quinoline | 9 | 15-16 |
| 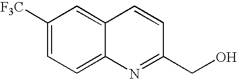 | (6-Trifluoromethyl- quinolin-2-yl)-methanol | 10 | 18 |
| 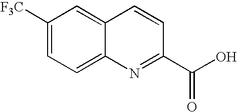 | 6-Trifluoromethyl- quinoline-2-carboxylic acid | 11 | 34-35 |
| 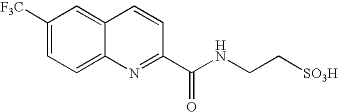 | 2-[(6-Trifluoromethyl- quinoline-2-carbonyl)- amino]-ethanesulfonic acid | 12 | 10-11 |
| 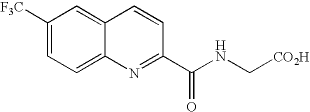 | [(6-Trifluoromethyl- quinoline-2-carbonyl)- amino]-acetic acid | 13 | 17.5 |
| 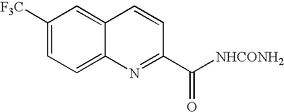 | (6-Trifluoromethyl- quinoline-2-carbonyl)-urea | 14 | 40 |
| 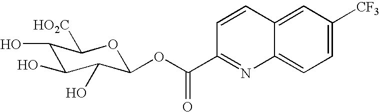 | 6-Trifluoromethyl- quinoline-2-carboxylic acid 6-carboxy-3,4,5-trihydroxy- tetrahydro-pyran-2-yl ester | 15 | 11-17.6 |
| 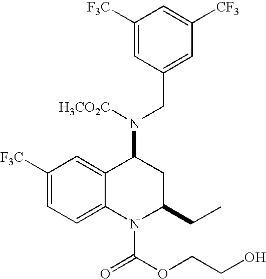 | 4-[(3,5-Bis-trifluoromethyl- benzyl)-methoxycarbonyl- amino]-2-ethyl-6- trifluoromethyl-3,4-dihydro- 2H-quinoline-1-carboxylic acid 2-hydroxy-ethyl ester | 16 | 46-47 |
| 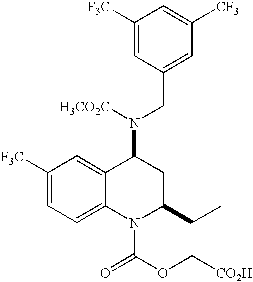 | 4-[(3,5-Bis-trifluoromethyl- benzyl)-methoxycarbonyl- amino]-2-ethyl-6- trifluoromethyl-3,4-dihydro- 2H-quinoline-1-carboxylic acid carboxymethyl ester | 17 | 46-47 |
| 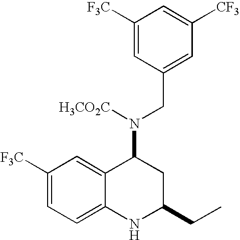 | (3,5-Bis-trifluoromethyl- benzyl)-(2-ethyl-6- trifluoromethyl-1,2,3,4- tetrahydro-quinolin-4-yl)- carbamic acid methyl ester | 23 | 49-50 |
| 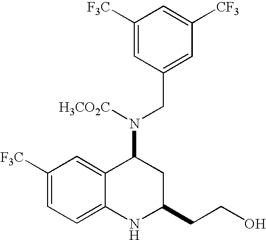 | (3,5-Bis-trifluoromethyl- benzyl)-[2-(2-hydroxy- ethyl)-6-trifluoromethyl- 1,2,3,4-tetrahydro-quinolin- 4-yl]-carbamic acid methyl ester | 24 | 45-48 |
| 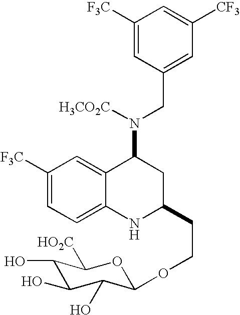 | 6-(2-{4-[(3,5-Bis- trifluoromethyl-benzyl)- methoxycarbonyl-amino]-6- trifluoromethyl-1,2,3,4- tetrahydro-quinolin-2-yl}- ethoxy)-3,4,5-trihydroxy- tetrahydro-pyran-2- carboxylic acid | 25 | 27 |
| 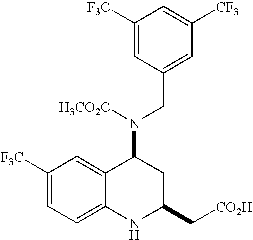 | {4-[(3,5-Bis- trifluoromethyl-benzyl)- methoxycarbonyl-amino]-6- trifluoromethyl-1,2,3,4- tetrahydro-quinolin-2-yl}- acetic acid | 26 | 45-46 |
| 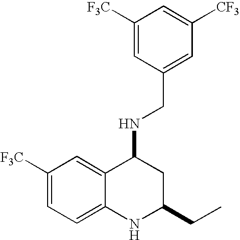 | (3,5-Bis-trifluoromethyl- benzyl)-(2-ethyl-6- trifluoromethyl-1,2,3,4- tetrahydro-quinolin-4-yl)- amine | 28 | 44 |
| 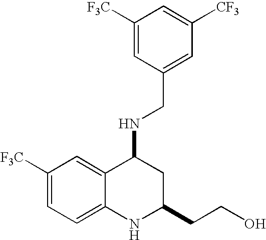 | 2-[4-(3,5-Bis- trifluoromethyl- benzylamino)-6- trifluoromethyl-1,2,3,4- tetrahydro-quinolin-2-yl]- ethanol | 29 | 42-43 |

Table 3 provides a list of metabolites of torcetrapib that were not specifically identified by the HPLC/mass spectroscopic metabolic profile. These compounds are expected to have a very short metabolic half-life, and thus, the compounds in the plasma, urine or feces are very difficult to detect. Nevertheless, these compounds can be identified as metabolites of torcetrapib because of their association with the metabolic pathways made evident by the compounds identified in Table 2 or are likely metabolic precursors or end-products to the products in Table 2.

TABLE 3

| | | |
|---|---|---|
| Compound Structure | Compound Name | No. |

| | | |
|---|---|---|
| 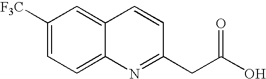 | (6-Trifluoromethyl- quinolin-2-yl)- acetic acid | 8 |
| 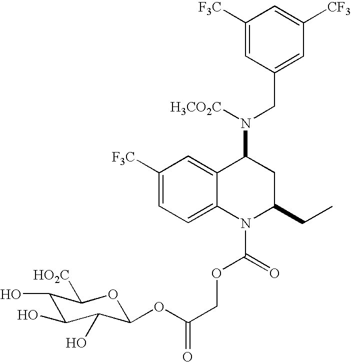 | 4-[(3,5-Bis- trifluoromethyl- benzyl)-methoxy- carbonyl-amino]- 2-ethyl-6-tri- fluoromethyl-3,4- dihydro-2H- quinoline-1- carboxylic acid 6- carboxy-3,4,5- trihydroxy-tetra- hydro-pyran-2- yloxycarbonyl- methyl ester | 18 |
| 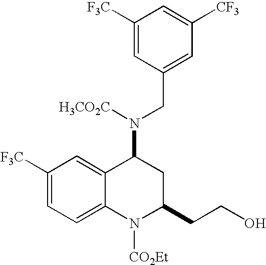 | 4-[(3,5-Bis- trifluoromethyl- benzyl)-methoxy- carbonyl-amino]- 2-(2-hydroxy- ethyl)-6-trifluoro- methyl-3,4-dihydro- 2H-quinoline-1- carboxylic acid ethyl ester | 19 |
| 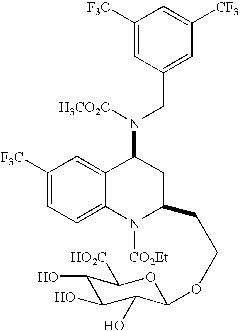 | 4-[(3,5-Bis- trifluoromethyl- benzyl)-methoxy- carbonyl-amino]- 2-[2-(6-carboxy- 3,4,5-trihydroxy- tetrahydro-pyran-2- yloxy)-ethyl]-6- trifluoromethyl-3,4- dihydro-2H- quinoline-1- carboxylic acid ethyl ester | 20 |
| 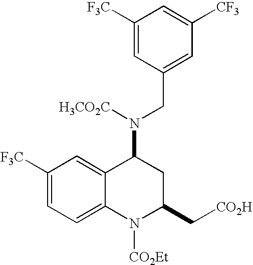 | 4-[(3,5-Bis- trifluoromethyl- benzyl)-methoxy- carbonyl-amino]- 2-carboxymethyl- 6-trifluoromethyl- 3,4-dihydro-2H- quinoline-1- carboxylic acid ethyl ester | 21 |
| 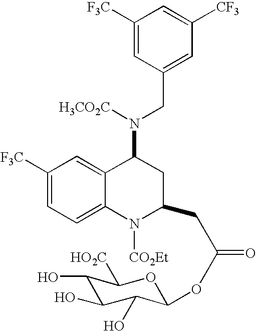 | 4-[(3,5-Bis- trifluoromethyl- benzyl)-methoxy- carbonyl-amino]- 2-(6-carboxy-3,4,5- trihydroxy-tetra- hydro-yloxy- carbonylmethyl)-6- trifluoromethyl-3,4- dihydro-2H- quinoline-1- carboxylic acid ethyl ester | 22 |
| 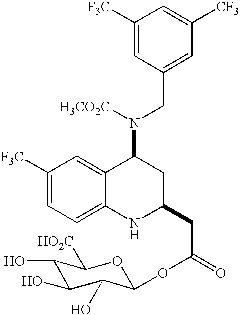 | 6-(2-{4-[(3,5-Bis- trifluoromethyl- benzyl)-methoxy- carbonyl-amino]-6- trifluoromethyl- 1,2,3,4-tetrahydro- quinolin-2-yl}- acetoxy)-3,4,5- trihydroxy- tetrahydro-pyran-2- carboxylic acid | 27 |
| 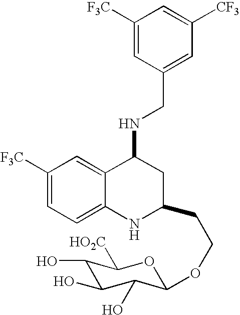 | 6-{2-[4-(3,5-Bis- trifluoromethyl- benzylamino)-6- trifluoromethyl- 1,2,3,4-tetrahydro- quinolin-2-yl]- ethoxy}-3,4,5- trihydroxy- tetrahydro-pyran-2- carboxylic acid | 30 |
| 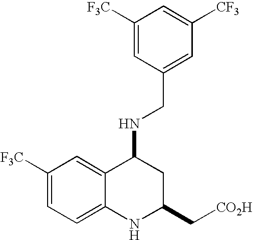 | [4-(3,5-Bis- trifluoromethyl- benzylamino)-6- trifluoromethyl- 1,2,3,4-tetrahydro- quinolin-2-yl]- acetic acid | 31 |
| 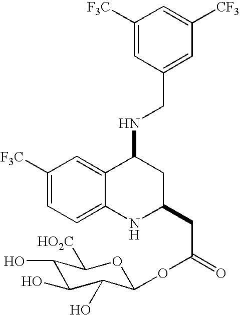 | 6-{2-[4-(3,5-Bis- trifluoromethyl- benzylamino)-6- trifluoromethyl- 1,2,3,4-tetrahydro- quinolin-2-yl]- acetoxy}-3,4,5- trihydroxy- tetrahydro-pyran-2- carboxylic acid | 32 |
| 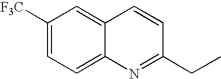 | (6-Trifluoromethyl- quinolin-2-yl)- ethyl | 33 |
| 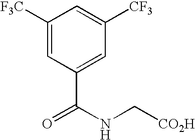 | (3,5-Bis- trifluoromethyl- benzoylamino)- acetic acid | 34 |

2. Synthetic Procedures for Selected Compounds Listed in Tables 2 and 3.

As used herein, the expressions “reaction-inert solvent” and “inert solvent” refers to a solvent or a mixture thereof which does not interact with starting materials, reagents, intermediates or products in a manner which adversely affects the yield of the desired product.

The terms “concentrated” and “evaporated” refer to removal of solvent at water aspirator pressure on a rotary evaporator with a bath temperature of less than 45° C. Reactions conducted at 0-20° C.′ or 0-25° C.′ were conducted with initial cooling of the vessel in an insulated ice bath which was allowed to warm to room temperature over several hours.

In accordance with reaction Schemes 1 to 4, a select number of compounds recited in Table 2 and Table 3 were prepared according to the following synthetic procedures. Many of the the compounds of Table 2 and Table 3 whose synthesis are not described in detail below can be prepared by those skilled in the art starting from one of the compounds provided below and using synthetic procedures well known to those of ordinary skill in the art.

An alternative synthetic route to the general class of [2R, 4S] 4-[(3,5-bis-trifluoromethyl-benzyl)-amino]-2-ethyl-6-trifluoromethyl-3,4-dihydro-2H-quinolines and analogs thereof is described in U.S. patent application Ser. No. 10/137,314 and U.S. Pat. No. 6,197,786, of which the entire disclosures are incorporated herein by reference.

### 6-Trifluoromethyl-quinoline-2-carboxylic acid (Compound 11)

4-Trifluoromethyl-aniline (2.0 g, 12.4 mmol), n-butyl glyoxaldehyde (1.8 g, 13.7 mmol) and anhydrous sodium sulfate (2.5 g) was combined in 150 mL of dichloromethane. After stirring for 1 hour and filtering through Celite, the concentrated imine was dissolved in toluene and combined with 1-bromo-2-ethoxy-ethene (3.4 g, 22.4 mmol) and p-toluenesulfonic acid monohydrate (236 mg, 1.24 mmol). After stirring at reflux for 6 hours, the reaction mixture was diluted with ethyl acetate, washed with a saturated NaHCO3 solution, dried over magnesium sulfate, filtered and concentrated to give a residue which was purified by chromatography on silica gel eluting with 0-10% ethyl acetate in hexanes to afford 1.5 g of 3-bromo-4-ethoxy-6-trifluoromethyl-1,2,3,4-tetrahydro-quinoline-2-carboxylic acid butyl ester.

3-Bromo-4-ethoxy-6-trifluoromethyl-1,2,3,4-tetrahydro-quinoline-2-carboxylic acid butyl ester (1.5 g, 3.54 mmol) was dissolved in 50 mL of THF and treated with DBU (1.0 mL). After 40 min, the reaction mixture was concentrated, taken up in ethyl acetate, and washed twice with 2N HCl. The organic phase was dried over magnesium sulfate, filtered and concentrated. The residue was purified by chromatography on silica, eluting with 5% ethyl acetate in hexanes to give 800 mg of 6-trifluoromethyl-quinoline-2-carboxylic acid butyl ester.

6-Trifluoromethyl-quinoline-2-carboxylic acid butyl ester (300 mg, 1.0 mmol) was dissolved in 10 mL of methanol and treated with 1.0 mL of a 2N NaOH solution. After stirring overnight, the volatiles were evaporated and the aqueous phase extracted with ethyl acetate. The aqueous phase was acidified with 1N HCl and extracted twice with ethyl acetate. The combined organic layers were dried over magnesium sulfate, filtered and concentrated to give the title compound as a colorless solid.

LCMS (ESI+): 242 (MH⁺). ¹H NMR (CDCl₃) δ 8.08 (1H, dd, J=8.7, 1.8 Hz), 8.31 (1H, d, J=8.3 Hz), 8.36 (1H, d, J=8.7 Hz), 8.48 (1H, s), 8.67 (1H, d, J=8.3 Hz).

### (6-Trifluoromethyl-quinolin-2-yl)-methanol (Compound 10)

6-Trifluoromethyl-quinoline-2-carboxylic acid butyl ester (100 mg, 0.35 mmol) was dissolved in 5 mL of methanol and sodium borohydride (57 mg, 1.57 mmol) was added. After 4 hours, the volatiles were evaporated and the residue dissolved in water and extracted with ethyl acetate. The combined organic phases were dried over magnesium sulfate, filtered and concentrated to provide the title compound.

¹H NMR (CDCl₃) δ 4.97 (2H, s), 7.42 (1H, d, J=8.7 Hz), 7.91 (1H, dd, J=8.8, 2.1 Hz), 8.16 (1H, s), 8.21 (1H, d, J=9.1 Hz), 8.25 (1H, d, J=8.7 Hz)

### 2-Methyl-6-trifluoromethyl-quinoline (Compound 9)

(6-Trifluoromethyl-quinolin-2-yl)-methanol (80 mg, 0.35 mmol) was dissolved in dichloromethane, cooled in an ice/water bath and treated with triethylamine (89 mg, 0.88 mmol) and acetyl chloride (55 mg, 0.71 mmol). After 1 hr, the cooling bath was removed and the mixture stirred at room temperature. After 4 hours, the reaction mixture was diluted with more dichloromethane, washed with 1N HCl, twice with saturated sodium bicarbonate solution, and brine. The organic phase was dried over magnesium sulfate, filtered, and concentrated to give acetic acid 6-trifluoromethyl-quinolin-2-ylmethyl ester.

Acetic acid 6-trifluoromethyl-quinolin-2-ylmethyl ester (50 mg) was dissolved in

5 mL of ethanol and 5 mL of cyclohexene, and treated with 10% palladium on carbon

(10 mg, 50% water weight). After heating at reflux for 4 hours, the cooled mixture was filtered through Celite, concentrated and the residue purified by chromatography on silica eluting with 5% ethyl acetate in hexanes to afford the title compound.

LCMS (EI): 211 (M). ¹H NMR (CD₃OD) δ 2.83 (3H, s), 7.69 (1H, d, J=8.3 Hz), 8.03 (1H, dd, J=8.8, 1.9 Hz), 8.16 (1H, d, J=9.1 Hz), 8.41 (1H, s), 8.57 (1H, d, J=8.7 Hz).

### (3,5-Bis-trifluoromethyl-phenyl)-methoxycarbonylamino-methanesulfonic acid (Compound 4)

3,5-Bis-trifluoromethyl-benzaldehyde (1.47 g, 6.1 mmol) and bis(ammonium)sulfinate were combined in 10 mL of water and heated at 50° C. After 2 hours, the reaction mixture was cooled in an ice/water bath and concentrated HCl was added to form a precipitate. After stirring for 10 min, the solid was collected by filtration, washed with 0.1 N HCl, ether, and air dried to yield 1.51 g of amino-(3,5-bis-trifluoromethyl-phenyl)-methanesulfonic acid.

Amino-(3,5-bis-trifluoromethyl-phenyl)-methanesulfonic acid (110 mg, 0.34 mmol) was dissolved in 2 mL of water containing 100 mg of potassium carbonate and treated with methyl chloroformate (30 μL, 0.34 mmol). After stirring for 1 hour, the precipitate formed was collected by filtration and air dried to afford 93 mg of the title compound, presumably as its potassium salt.

¹H NMR (CD₃OD) δ 3.69 (3H, s), 5.82 (1H, s), 7.89 (1H, s), 8.18 (2H, s)

### 3,5-bis-trifluoromethyl-benzyl)-[2-(2-hydroxy-ethyl)-6-trifluoromethyl-1,2,3,4-tetrahydro-quinolin-4-yl]-(carbamic acid methyl ester (Compound 24)

In accordance with Scheme 1, a mixture of 4-trifluoromethyl-aniline (5 g, 31 mmol) and 1H-benzotriazole (3.7 g, 31 mmol) in 50 mL of toluene was added to a solution of 3-benzyloxy-propionaldehyde (5.1 g, 31 mmol) in 50 mL of toluene. After stirring at room temperature for 2 hours, the mixture was concentrated and the residue tritrated with hexanes to obtain (1-Benzotriazol-1-yl-3-benzyloxy-propyl)-(4-trifluoromethyl-phenyl)-amine.

(1-Benzotriazol-1-yl-3-benzyloxy-propyl)-(4-trifluoromethyl-phenyl)-amine (5.0 g, 11.76 mmol) and vinyl-carbamic acid benzyl ester (2.1 g, 11.76 mmol) were dissolved in 50 mL of dichloromethane and cooled to −15° C. Borontrifluoride etherate (167 mg, 1.18 mmol) was then added. After 2 hours at this temperature and 1 hour at room temperature, the reaction mixture was diluted with dichloromethane, washed with a saturated sodium bicarbonate solution, then brine, and dried over magnesium sulfate. The solution was then filtered and concentrated. The residue was purified by chromatography on silica eluting with 5-10% ethyl acetate in hexanes to give [2-(2-Benzyloxy-ethyl)-6-trifluoromethyl-1,2,3,4-tetrahydro-quinolin-4-yl]-carbamic acid benzyl ester.

[2-(2-Benzyloxy-ethyl)-6-trifluoromethyl-1,2,3,4-tetrahydro-quinolin-4-yl]-carbamic acid benzyl ester (2.0 g, 4.24 mmol) and pyridine (0.86 mL, 10.6 mmol) were combined in 40 mL of dichloromethane and cooled in an ice/water bath as trifluoroacetic anhydride (0.72 mL, 5.1 mmol) was added. After 2 hours the cooling bath was removed and after 1 hour further, the reaction mixture was washed with two portions of 1N HCl, saturated sodium bicarbonate solution, then brine, and dried over magnesium sulfate. The solution was filtered and concentrated to provide [2-(2-Benzyloxy-ethyl)-1-(2,2,2-trifluoro-acetyl)-6-trifluoromethyl-1,2,3,4-tetrahydro-quinolin-4-yl]-carbamic acid benzyl ester.

[2-(2-Benzyloxy-ethyl)-1-(2,2,2-trifluoro-acetyl)-6-trifluoromethyl-1,2,3,4-tetrahydro-quinolin-4-yl]-carbamic acid benzyl ester (2.0 g, 3.45 mmol) in 60 mL of ethanol was combined with 10% palladium on carbon (300 mg, 50% wet wt.) and shaken under 50 psi of hydrogen gas in a Parr bottle. After 1 hour, the reaction mixture was filtered through Celite, concentrated and purified by chromatography on silica eluting with 50% ethyl acetate in hexanes to give 1-[4-Amino-2-(2-benzyloxy-ethyl)-6-trifluoromethyl-3,4-dihydro-2H-quinolin-1-yl]-2,2,2-trifluoro-ethanone.

1-[4-Amino-2-(2-benzyloxy-ethyl)-6-trifluoromethyl-3,4-dihydro-2H-quinolin-1-yl]-2,2,2-trifluoro-ethanone (1.0 g, 2.45 mmol) was combined with 3,5-bis-trifluoromethyl-benzaldehyde (0.4 mL, 2.45 mmol) in 70 mL of 1,2-dichloroethane. After 1.5 hours, the reaction mixture was treated with sodium triacetoxyborohydride

(2.6 g, 12.25 mmol) and stirred overnight before adding an aqueous 2N KOH solution. The organic layer is separated, dried over magnesium sulfate, filtered and concentrated. The resulting residue was purified by chromatography on silica eluting with a 10% ethyl acetate in hexanes solution to provide 1-[2-(2-Benzyloxy-ethyl)-4-(3,5-bis-trifluoromethyl-benzylamino)-6-trifluoromethyl-3,4-dihydro-2H-quinolin-1-yl]-2,2,2-trifluoro-ethanone.

1-[2-(2-Benzyloxy-ethyl)-4-(3,5-bis-trifluoromethyl-benzylamino)-6-trifluoromethyl-3,4-dihydro-2H-quinolin-1-yl]-2,2,2-trifluoro-ethanone (1 g, 1.49 mmol) was dissolved in 25 mL of dichloromethane and cooled in an ice/water bath as pyridine (1 mL, 12.4 mmol) and methyl chloroformate (1 mL, 12.9 mmol) were added. After stirring overnight at room temperature, the reaction mixture was extracted with 2N HCl (twice), a saturated sodium bicarbonate solution, and brine then dried over magnesium sulfate, filtered and concentrated. The residue was purified by chromatography on silica eluting with a 5% then a 10% ethyl acetate in hexanes solution to provide [2-(2-Benzyloxy-ethyl)-1-(2,2,2-trifluoro-acetyl)-6-trifluoromethyl-1,2,3,4-tetrahydro-quinolin-4-yl]-(3,5-bis-trifluoromethyl-benzyl)-carbamic acid methyl ester.

[2-(2-Benzyloxy-ethyl)-1-(2,2,2-trifluoro-acetyl)-6-trifluoromethyl-1,2,3,4-tetrahydro-quinolin-4-yl]-(3,5-bis-trifluoromethyl-benzyl)-carbamic acid methyl ester (1 g, 1.37 mmol) was dissolved in 25 mL of a solution consisting of methanol, tetrahydrofuran and water in a 3:1:1 ratio and treated with a 1N LiOH solution (10 mL, 10 mmol). After stirring for a day, the volatiles were evaporated and the aqueous phase extracted with ethyl acetate. The combined organic phases were dried over magnesium sulfate, filtered and concentrated. The resulting residue was purified by chromatography on silica eluting with a 10% ethyl acetate in hexanes solution to provide [2-(2-Benzyloxy-ethyl)-6-trifluoromethyl-1,2,3,4-tetrahydro-quinolin-4-yl]-(3,5-bis-trifluoromethyl-benzyl)-carbamic acid methyl ester.

[2-(2-Benzyloxy-ethyl)-6-trifluoromethyl-1,2,3,4-tetrahydro-quinolin-4-yl]-(3,5-bis-trifluoromethyl-benzyl)-carbamic acid methyl ester (700 mg, 1.10 mmol) was dissolved in 50 mL of anhydrous ethanol and shaken in a Parr bottle under 50 psi of hydrogen gas. After 4 h, the reaction mixture was filtered through Celite, concentrated and the resulting residue purified by chromatography on silica eluting with a 10% ethyl acetate in hexanes solution to provide the title compound.

LCMS (ESI+): 545 (MH+).

### 4-[(3,5-Bis-trifluoromethyl-benzyl)-methoxycarbonyl-amino]-2-(2-hydroxy-ethyl)-6-trifluoromethyl-3,4-dihydro-2H-quinoline-1-carboxylic acid ethyl ester (Compound 19)

(3,5-Bis-trifluoromethyl-benzyl)-[2-(2-hydroxy-ethyl)-6-trifluoromethyl-1,2,3,4-tetrahydro-quinolin-4-yl]-carbamic acid methyl ester (500 mg, 0.92 mmol) was dissolved in 10 mL of anhydrous dimethylformamide and combined with imidazole (125 mg, 1.8 mmol) and tert-butyldimethylsilyl chloride (280 mg, 1.8 mmol). After stirring overnight, the reaction mixture was combined with water and extracted with ethyl acetate. The combined organics were dried over magnesium sulfate, filtered and concentrated to provide (3,5-Bis-trifluoromethyl-benzyl)-{2-[2-(tert-butyl-dimethyl-silanyloxy)-ethyl]-6-trifluoromethyl-1,2,3,4-tetrahydro-quinolin-4-yl)}-carbamic acid methyl ester.

(3,5-Bis-trifluoromethyl-benzyl)-{2-[2-(tert-butyl-dimethyl-silanyloxy)-ethyl]-6-trifluoromethyl-1,2,3,4-tetrahydro-quinolin-4-yl}-carbamic acid methyl ester (500 mg, 0.76 mmol) was dissolved in 250 ml of dichloromethane, and cooled in an ice/water bath as pyridine (0.5 mL) and ethyl chloroformate (1.0 mL) are added. After stirring for 1 h, the ice bath was removed and the reaction mixture allowed to warm to room temperature. After stirring overnight, the reaction mixture was washed with 2 N HCl, a saturated sodium bicarbonate solution, and brine before the organic phase was dried over magnesium sulfate, filtered and concentrated. The residue was purified by chromatography on silica eluting with a 5% then a 10% ethyl acetate in hexanes solution to provide 4-[(3,5-Bis-trifluoromethyl-benzyl)-methoxycarbonyl-amino]-2-[2-(tert-butyl-dimethyl-silanyloxy)-ethyl]-6-trifluoromethyl-3,4-dihydro-2H-quinoline-1-carboxylic acid ethyl ester.

4-[(3,5-Bis-trifluoromethyl-benzyl)-methoxycarbonyl-amino]-2-[2-(tert-butyl-dimethyl-silanyloxy)-ethyl]-6-trifluoromethyl-3,4-dihydro-2H-quinoline-1-carboxylic acid ethyl ester (200 mg) was dissolved in 10 mL of tetrahydrofuran and treated with tetrabutylammonium fluoride (1 mL of a 1 molar solution in tetrahydrofuran). After 1 h, the reaction mixture was concentrated, the residue partitioned between water and ethyl acetate, and the aqueous layer extracted further with ethyl acetate. The combined organic layers were dried over magnesium sulfate, filtered and concentrated. The residue was purified by chromatography on silica eluting with a 10% ethyl acetate in hexanes solution to provide the title compound.

### {4-[(3,5-Bis-trifluoromethyl-benzyl)-methoxycarbonyl-amino]-6-trifluoromethyl-1,2,3,4-tetrahydro-quinolin-2-yl}-acetic acid (Compound 21)

(3,5-Bis-trifluoromethyl-benzyl)-[2-(2-hydroxy-ethyl)-6-trifluoromethyl-1,2,3,4-tetrahydro-quinolin-4-yl]-carbamic acid methyl ester can be oxidized by one of a number of methods known to those skilled in the art (such as treatment of the alcohol as a solution in acetone with a chromic acid solution) to the desired carboxylic acid product.

### 4-[(3,5-Bis-trifluoromethyl-benzyl)-methoxycarbonyl-amino]-2-carboxymethyl-6-trifluoromethyl-3,4-dihydro-2H-quinoline-1-carboxylic acid ethyl ester (Compound 26)

4-[(3,5-Bis-trifluoromethyl-benzyl)-methoxycarbonyl-amino]-2-(2-hydroxy-ethyl)-6-trifluoromethyl-3,4-dihydro-2H-quinoline-1-carboxylic acid ethyl ester can be oxidized by one of a number of methods known to those skilled in the art (such as treatment of the alcohol as a solution in acetone with a chromic acid solution) to the desired carboxylic acid product.

Preparation of the Glycoside Compounds of the Invention by In Vitro Glucuronidation

A typical incubation mixture, in a final volume of 0.3 ml, contained 0.3 mg (dog) or 0.45 mg (human or rat) of liver microsomal protein, preincubated for 15 min with 0.045 mg of Brij 58, 20 mM MgCl, 5 mM UDPGA, and 0.05 M Tris buffer, pH 7.0. The preincubation step with Brij 58 was found to be optimal for high enzyme activity. Unless otherwise specified, the reaction was started by the addition of the appropriate substrate following a three minute preincubation at 37° C. Control experiments were performed by excluding either the microsomes or UDPGA from the incubation mixtures. The reaction was terminated at appropriate time intervals by the addition of 0.8 ml of acetonitrile (ACN). The ACN extracts were evaporated to dryness and reconstituted, just before analysis, in the mobile phase (20% ACN in 25 mM ammonium acetate buffer, pH 4.5) for analysis by the HPLC method described above.

Incubations with human recombinant UGTs were performed using the same conditions as described above for human liver microsomes, except that the mixture contained 0.3 mg of UGTs and was incubated for up to sixty minutes at 37° C. Control incubations using microsomes isolated from the same cell line containing the vector, but without a cDNA insert, also were included.

For the purpose of isolation and purification of the glucuronides, large-scale incubations of the compounds (100 μM; 20×0.5-ml incubation) can be carried out with dog liver microsomes (2 mg/ml) and UDPGA (5 mM) for 60 min. The ACN extracts are evaporated to dryness and reconstituted for analysis by LC-MS and LC-NMR spectroscopy.

We claim:

1. A compound having the Formula I 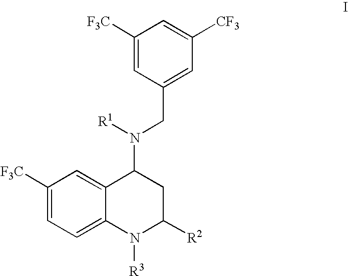
wherein
R¹ is —CO₂CH₃ or —H;
R² is —CH₂CH₃, —CH₂CH₂OH, —CH₂CO₂H, —CH₂CO₂A, and —CH₂CH₂OA, wherein A is 3,4,5-trihydroxy-tetrahydropyran-2-carboxylic acid; and
R³ is —H, —CO₂CH₂CH₃, —CO₂CH₂CH₂OH, —CO₂CH₂CO₂H, —CO₂CH₂CH₂OA and —CO₂CH₂CO₂A; or a pharmaceutically acceptable salt of said compound with the proviso that
if R¹ is —CO₂CH₃ and R³ is —H, then R² is not —CH₂CH₃, —CH₂CH₂OH, or —CH₂CO₂H;
if R¹ is —CO₂CH₃ and R³ is —CO₂CH₂CH₃, then R² is not —CH₂CH₂, —CH₂CH₂OH, or —CH₂CO₂H; and
if R¹ is —CO₂CH₃ and R² is —CH₂CH₃, then R³ is not —CO₂CH₂CH₂OH, or —CO₂CH₂CO₂H.

2. The compound of claim 1 wherein R¹ is —CO₂CH₃, R³ is CO₂CH₂CH₃, and R² is selected from —CH₂CO₂A or —CH₂CH₂OA.

3. The compound of claim 1 wherein R¹ is —CO₂CH₃, R³ is —H, and R² is selected from —CH₂CO₂A or —CH₂CH₂OA.

4. The compound of claim 1 wherein R¹ and R³ is H, and R² is selected from the group consisting of —CH₂CH₃, —CH₂CH₂OH, —CH₂CO₂H, —CH₂CO₂A, and CH₂CH₂OA.

5. The compound of claim 1 wherein R¹ is —CO₂CH₃, R² is —CH₂CH₃, and R³ is —CO₂CH₂CO₂A.

6. A compound selected from the group consisting of
[2R, 4S] 4-[(3,5-bis-trifluoromethyl-benzyl)-methoxycarbonyl-amino]-2-ethyl-6-trifluoromethyl-3,4-dihydro-2H-quinoline-1-carboxylic acid 2-hydroxyethyl ester;
[2R, 4S] 4-[(3,5-bis-trifluoromethyl-benzyl)-methoxycarbonyl-amino]-2-ethyl-6-trifluoromethyl-3,4-dihydro-2H-quinoline-1-carboxylic acid carboxymethyl ester;
[2R, 4S] 4-[(3,5-bis-trifluoromethyl-benzyl)-methoxycarbonyl-amino]-2-carboxymethyl-6-trifluoromethyl-3,4-dihydro-2H-quinoline-1-carboxylic acid ethyl ester;
[2R, 4S] 4-[(3,5-bis-trifluoromethyl-benzyl)-(2-ethyl-6-trifluoromethyl-1,2,3,4-tetrahydro-quinolin-4-yl)-carbamic acid methyl ester;
[2R, 4S] 4-[(3,5-bis-trifluoromethyl-benzyl)-[2-(2-hydroxyethyl)-6-trifluoromethyl-1,2,3,4-tetrahydro-quinolin-4-yl]-carbamic acid methyl ester; and
[2R, 4S] {4-[(3,5-bis-trifluoromethyl-benzyl)-methoxycarbonyl-amino]-6-trifluoromethyl-1,2,3,4-tetrahydro-quinolin-2-yl}-acetic acid.

7. A compound of Formula II 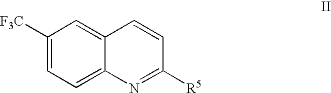
wherein R⁵ is —CH₂CH₃, —CO₂H, —CO₂A, —CH₂CH₂OH, —CH₂CO₂H, —CH₂CH₂OA, —CH₂CH₂OSO₃H, —C(O)N(H)CH₂CH₂SO₃H, —C(O)N(H)CH₂CO₂H, and —C(O)N(H)C(O)NH₂, and wherein A is 3,4,5-trihydroxy-tetrahydropyran-2-carboxylic acid.

8. The compound of claim 7 wherein R⁵ is selected from —CH₂CH₃ or —CO₂H.

9. A compound of Formula III 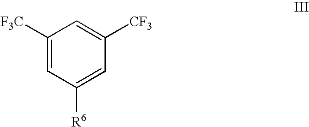
wherein R⁶ is —CH₂OA, —C(O)N(H)CH₂CO₂A and —CH(SO₃H)N(H)CO₂CH₃, and wherein A is 3,4,5-trihydroxy-tetrahydropyran-2-carboxylic acid.

10. A method for indicating the presence of or exposure to 4-[(3,5-bis-trifluoromethyl-benzyl)-methoxycarbonyl-amino]-2-ethyl)-6-trifluoromethyl-3,4-dihydro-2H-quinoline-1-carboxylic acid ethyl ester in a mammal comprising monitoring the presence of a compound of claim 1 in the mammal.

11. A method for indicating the presence of or exposure to 4-[(3,5-bis-trifluoromethyl-benzyl)-methoxycarbonyl-amino]-2-ethyl)-6-trifluoromethyl-3,4-dihydro-2H-quinoline-1-carboxylic acid ethyl ester in a mammal comprising monitoring the presence of a compound of claim 6 or 4-[(3,5-bis-trifluoromethyl-benzyl)-methoxycarbonyl-amino]-(2-hydroxy-ethyl)-6-trifluoromethyl-3,4-dihydro-2H-quinoline-1-carboxylic acid ethyl ester in the mammal.

12. A method for indicating the presence of or exposure to 4-[(3,5-bis-trifluoromethyl-benzyl)-methoxycarbonyl-amino]-2-ethyl)-6-trifluoromethyl-3,4-dihydro-2H-quinoline-1-carboxylic acid ethyl ester in a mammal comprising monitoring the presence of a compound selected from the group consisting of a compound of
claim 7, 2-methyl-6-trifluoromethyl-quinoline, and (6-trifluoromethyl-quinolin-2-yl)methanol in the mammal.

13. A method for indicating the presence of or exposure to 4-[(3,5-bis-trifluoromethyl-benzyl)-methoxycarbonyl-amino]-2-ethyl)-6-trifluoromethyl-3,4-dihydro-2H-quinoline-1-carboxylic acid ethyl ester in a mammal comprising monitoring the presence of a compound selected from the group consisting of 3,5-Bis-trifluoromethyl-benzoic acid, 6-(3,5-Bis-trifluoromethyl-benzoyloxy)-3,4,5-trihydroxy-tetrahydro-pyran-2-carboxylic acid, 6-(3,5-Bis-trifluoromethyl-benzyloxy)-3,4,5-trihydroxy-tetrahydro-pyran-2-carboxylic acid, (3,5-Bis-trifluoromethyl-phenyl)-methoxycarbonylamino-methanesulfonic acid, (3,5-Bis-trifluoromethyl-benzoylamino)-acetic acid, and (3,5-Bis-trifluoromethyl-benzoylamino)-3,4,5-trihydroxy-tetrahydro-pyran-2-carboxylic acid in the mammal.

14. A method for indicating the presence of or exposure to 4-[(3,5-bis-trifluoromethyl-benzyl)-methoxycarbonyl-amino]-2-ethyl)-6-trifluoromethyl-3,4-dihydro-2H-quinoline-1-carboxylic acid ethyl ester in a mammal comprising monitoring the presence of a compound selected from the group consisting of 3,5-bis-trifluoromethylbenzoic acid, 2-methyl-6-trifluoromethyl-quinoline, and 6-trifluoromethyl-quinoline-2-carboxylic acid in the mammal.

15. A method for treating atherosclerosis comprising administering to a mammal an atherosclerosis treating amount of a compound of Formula I 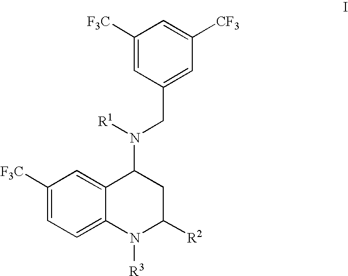
wherein
R¹ is —CO₂CH₃ or —H;
R² is —CH₂CH₃, —CH₂CH₂OH, —CH₂CO₂H, —CH₂CO₂A, and —CH₂CH₂OA, wherein A is 3,4,5-trihydroxy-tetrahydropyran-2-carboxylic acid; and
R³ is —H, —CO₂CH₂CH₃, —CO₂CH₂CH₂OH, —CO₂CH₂CO₂H, —CO₂CH₂CH₂OA and —CO₂CH₂CO₂A; a prodrug thereof, or a pharmaceutically acceptable salt of said compound or of said prodrug with the proviso that
if R¹ is —CO₂CH₃ and R³ is —H, then R² is not —CH₂CH₃, —CH₂CH₂OH, or —CH₂CO₂H;
if R¹ is —CO₂CH₃ and R³ is —CO₂CH₂CH₃, then R² is not —CH₂CH₂, —CH₂CH₂OH, or —CH₂CO₂H; and
if R¹ is —CO₂CH₃ and R² is —CH₂CH₃, then R³ is not —CO₂CH₂CH₂OH, or —CO₂CH₂CO₂H, or a compound a compound selected from the group consisting of
[2R, 4S] 4-[(3,5-bis-trifluoromethyl-benzyl)-methoxycarbonyl-amino]-2-ethyl-6-trifluoromethyl-3,4-dihydro-2H-quinoline-1-carboxylic acid 2-hydroxyethyl ester;
[2R, 4S] 4-[(3,5-bis-trifluoromethyl-benzyl)-methoxycarbonyl-amino]-2-ethyl-6-trifluoromethyl-3,4-dihydro-2H-quinoline-1-carboxylic acid carboxymethyl ester;
[2R, 4S] 4-[(3,5-bis-trifluoromethyl-benzyl)-methoxycarbonyl-amino]-2-carboxymethyl-6-trifluoromethyl-3,4-dihydro-2H-quinoline-1-carboxylic acid ethyl ester;
[2R, 4S] 4-[(3,5-bis-trifluoromethyl-benzyl)-(2-ethyl-6-trifluoromethyl-1,2,3,4-tetrahydro-quinolin-4-yl)-carbamic acid methyl ester;
[2R, 4S] 4-[(3,5-bis-trifluoromethyl-benzyl)-[2-(2-hydroxyethyl)-6-trifluoromethyl-1,2,3,4-tetrahydro-quinolin-4-yl]-carbamic acid methyl ester;
[2R, 4S] {4-[(3,5-bis-trifluoromethyl-benzyl)-methoxycarbonyl-amino]-6-trifluoromethyl-1,2,3,4-tetrahydro-quinolin-2-yl}-acetic acid, and
a prodrug thereof, or a pharmaceutically acceptable amount salt of said compound or of said prodrug.
